(19) United States
(12) Patent Application Publication
(10) Pub. No.: US 2026/0219249 A1
(43) Pub. Date: Jul. 30, 2026

(54) SEQUENTIAL SENSOR PLACEMENT FOR GAS EMISSION DETECTION USING RECORD COUNT

(71) Applicant: Schlumberger Technology Corporation, Sugar Land, TX (US)

(72) Inventor: Kashif Rashid, Wayland, MA (US)

(21) Appl. No.: 19/008,919

(22) Filed: Jan. 3, 2025

(51) Int. Cl. G01N 33/00; G01M 3/04

(52) U.S. Cl. CPC G01N 33/0031; G01M 3/04; G01N 33/0047

(57) ABSTRACT

Aspects of the disclosure provide for sensor placement for gas emission detection using record count. A method for sensor placement includes obtaining wind rose distribution data associated with a site for gas emission detection and generating a plurality of wind realizations from the wind rose distribution data. The method includes generating a plurality of records associated with predicted sensor measurements at each candidate sensor location for each of a plurality of potential gas emission locations at the site and subject to each of the plurality of wind realizations and ranking the candidate sensor location based on a record count. The method includes iteratively selecting for gas emission detection sensor placement a candidate sensor location, from the plurality of candidate sensor locations, having a highest ranking and removing at least the selected candidate sensor location from the plurality of candidate sensor locations.

### BACKGROUND

### Field of the Disclosure

The disclosure relates to leak detection systems and, more particularly, to sensor placement for leak detection.

### Description of Related Art

Methane (CH₄) is a potent greenhouse pollutant predicted to account for over thirty percent (30%) of global warming over the next decade. Methane is considered to be between 50 to 84 times more potent than carbon dioxide in the atmosphere. The Intergovernmental Panel on Climate Change (IPCC) has recommended the reduction in anthropogenic methane emissions to limit global temperature rise to 1.5° C. above pre-industrial levels by the year 2030.

There is a growing need for the oil and gas industry to monitor assets for methane leaks in order to mitigate the source of emissions. Methane is a primary component of natural gas. Hence, the global oil and gas industry is one of the largest sources of anthropogenic methane emissions. The oil and gas industry is deemed responsible for over twenty percent (20%) of anthropogenic methane emissions released annually. These emissions can be categorized in two forms, as intentionally vented or unintentionally fugitive. Vented releases result from operational activity in which methane is released knowingly, such as a consequence of equipment use (e.g., pneumatic natural gas valves), due to routine inefficient flaring or worse, due to direct release of gas into the atmosphere due to lack of collection means during production, or resulting directly from well intervention procedures. Fugitive releases, on the other hand, are those that may result due to faulty or failed equipment such as wellheads, separators, compressors and pipelines, etc. For example, significant leaks can occur across the entire oil and gas value chain, from production and processing to transmission, storage, and distribution. The risk is compounded by aging and unmonitored assets comprising thousands of equipment items that may be a source of leak.

Detection and remediation of methane leaks is important to controlling and reducing methane emissions at oil and gas sites. It is desirable to quickly identify and repair methane pollution sources. Minimizing the time required to identify a leak and the subsequent time to dispatch repair crews can significantly reduce the amount of methane released into the atmosphere.

Methane detection and remediation has challenges. Methane is an invisible gas. A methane emission can come from leaking components anywhere in the infrastructure. Further, a substantial portion of the methane emissions from oil and gas facilities arise from events that are intermittent. In addition, weather conditions impacts the detection of methane gas.

One approach for methane detection is a continuous monitoring system. For example, a continuous monitoring system may use methane emission detectors installed at an oilfield facility to continuously monitor leaked gas concentration above background levels. Continuous monitoring systems can accurately measure emissions from sources that leak or vent intermittently. Examples of methane emission detectors include methane point sensors and methane light detection and ranging (LiDAR) cameras. One example of a methane point sensor is the methane point instrument from the SLB End-to-End Emissions Solution (SEES) provided by Schlumberger Limited, Houston, Texas. In some aspects, the continuous methane monitoring system uses Internet-of-Things (IoT) enabled sensors to quickly and cost effectively detect, locate, and quantify methane emissions. The methane point sensors may be used to detect when emissions start and stop, to triangulate to the emission source, and to quantify the emission rate. The continuous monitoring system may generate a picture that identifies the beginning of a methane emission plume, showing the emission source.

Sensor placement is an important consideration for a methane emission detection system. In current methane detection systems, optimization of methane sensor placement is computationally intensive. For example, sensor placement optimization may involve optimizing a stated objective function, to maximize coverage of potential leaks, minimize detection time, or maximize leak detection given prevailing wind patterns. A computational algorithm may test different configurations of sensor placements to find the most effective layout based on the optimization goal (e.g., based on the objective function). Inverse modeling techniques to predict the optimal sensor positions by backtracking from potential detection points to the likely source of emissions under various wind conditions. For example, data that the sensors collect as a function of time can be used to invert back to the leak source using triangulation, in conjunction with an appropriate forward model (e.g., the Gaussian Plume Model-a pollutant dispersant model). Using such approaches, given a known methane source (e.g., a candidate leak point), a leak rate, and wind conditions, a model can be created to estimate point concentration (e.g., a methane concentration detection at a candidate sensor location).

Given that wind conditions at a site are prone to variation, establishing an optimal sensor placement may need to consider many different potential wind realizations. With greater wind variability over time, a plume of leaked methane will meander in different directions hitting different sensor locations. The wind will carry methane away from the leak source, and the meandering plume will hit the fixed sensors when the conditions are favorable. The use of wind realizations helps to account for the wind uncertainty during the evaluation stage.

Point sensors collect methane concentration over time. In certain approaches, each point sensor collect methane detection measurements over a time period resulting in a set of records. For example, the methane concentration (in ppm) may be stored periodically (e.g., every 1 minute) and processed in a time window (e.g., every 5-10 minutes) to generate a set of records. Each may record may include a measured methane concentration, Cₚ, the wind realization (e.g., wind speed, w_{spd}, and wind direction, w_{dir}), and the sensor location (e.g., sₓ, s_{y}, s_{z}). The set of records can be used to solve an inverse problem to identify the likely source of a methane leak.

In current approaches, to optimize sensor placement, each sensor placement optimization may entail ne×nw inversions, where ne is the number of leak candidates on the site and nw is the number of wind realizations used. That is, to evaluate a coverage measure of a sensor placement optimization, an inversion is solved for each wind realization and each candidate sensor location for a number of iterations in the optimization algorithm. As the number of wind realizations and candidate sensors locations may be large, this can results in a very large computational overhead to perform the sensor placement optimization. While each inversion computation may take a number of seconds (e.g., ~3 secs), with ne=100 and nw=5, there will be 500 inversions requiring 1500 secs, or around 25 minutes for each objective evaluation. If the optimization process takes 200 iterations, the total time for the sensor placement optimization could be 200*25 mins=5000 mins or around 83 hours or 3.5 days.

Accordingly, there exists a need for further improvements in methane emission detection and, more particularly, for fast and efficient sensor placement.

### SUMMARY

The disclosure provides techniques for sensor placement for methane emission detection.

Some aspects provide a method for sensor placement. The method includes obtaining wind rose distribution data associated with a site for gas emission detection. The method includes generating a plurality of wind realizations from the wind rose distribution data. The method includes for each candidate sensor location of a plurality of candidate sensor locations at the site:
generating a plurality of records associated with predicted sensor measurements at the candidate sensor location for each of a plurality of potential gas emission locations at the site and subject to each of the plurality of wind realizations; generating a record count based on the plurality of records; and ranking the candidate sensor location based on the record count. The method includes iteratively selecting candidate sensor locations, from the plurality of candidate sensor locations, based on the ranking. The iteratively selecting includes, in each iteration: selecting for gas emission detection sensor placement a candidate sensor location, from the plurality of candidate sensor locations, having a highest ranking; and removing at least the selected candidate sensor location from the plurality of candidate sensor locations. The method includes placing sensors at the selected candidate sensor locations at the site for gas emission detection.

Some aspects provide an apparatus for automated sensor placement location selection. The apparatus includes one or more memories storing computer executable code. The apparatus includes one or more processors configured to execute the computer executable code and cause the apparatus to: obtain wind rose distribution data associated with a site for gas emission detection; generate a plurality of wind realizations from the wind rose distribution data; for each candidate sensor location of a plurality of candidate sensor locations at the site: generate a plurality of records associated with predicted sensor measurements at the candidate sensor location for each of a plurality of potential gas emission locations at the site and subject to each of the plurality of wind realizations; generate a record count based on the plurality of records; and rank the candidate sensor location based on the record count; iteratively select candidate sensor locations, from the plurality of candidate sensor locations, based on the ranking, wherein the iteratively selecting comprises, in each iteration: selecting for gas emission detection sensor placement a candidate sensor location, from the plurality of candidate sensor locations, having a highest ranking; and removing at least the selected candidate sensor location from the plurality of candidate sensor locations; and output the selected sensor locations.

Some aspects provide a computer readable medium storing computer executable code for automated sensor placement location selection. The computer executable code may include code for obtaining wind rose distribution data associated with a site for gas emission detection. The computer executable code may include code for code for generating a plurality of wind realizations from the wind rose distribution data. The computer executable code may include code for, for each candidate sensor location of a plurality of candidate sensor locations at the site, generating a plurality of records associated with predicted sensor measurements at the candidate sensor location for each of a plurality of potential gas emission locations at the site and subject to each of the plurality of wind realizations. The computer executable code may include code for, for each candidate sensor location of a plurality of candidate sensor locations at the site, generating a record count based on the plurality of records. The computer executable code may include code for, for each candidate sensor location of a plurality of candidate sensor locations at the site, ranking the candidate sensor location based on the record count. The computer executable code may include code for iteratively selecting candidate sensor locations, from the plurality of candidate sensor locations, based on the ranking. The code for iteratively selecting may include code for, in each iteration, selecting for gas emission detection sensor placement a candidate sensor location, from the plurality of candidate sensor locations, having a highest ranking; and code for, in each iteration, removing at least the selected candidate sensor location from the plurality of candidate sensor locations. The computer executable code may include code for outputting the selected sensor locations.

The following description and the appended figures set forth certain features for purposes of illustration.

### BRIEF DESCRIPTION OF DRAWINGS

The figures show several embodiments of the system according to the disclosure.

FIG. 1 illustrates an example gas emission detection site.

FIG. 2 illustrates an example wind rose.

FIG. 3 is a graph illustrating a wind rose distribution extracted from a wind rose.

FIG. 4A is a graph illustrating the record count per candidate sensor location for all selected wind realizations along with the mean record count per candidate sensor location.

FIG. 4B is a graph illustrating the mean record count per candidate sensor location at the candidate sensor locations.

FIGS. 5A-5G illustrate example sensor placement selection for the gas emission detection site.

FIGS. 6A-6C illustrate example sensor placement selection for the gas emission detection site with different separation distances.

FIGS. 7A-7D depict a flow diagram depicting an example method for sensor placement for gas emission detection using record count.

FIG. 8 is an example processing system for gas emission sensor placement selection using record count.

### DETAILED DESCRIPTION

The disclosure provides techniques, methods, systems, apparatus, and computer readable media for sensor placement for emission detection with wind uncertainty.

It should be understood that while aspects of the present disclosure are described with respect to methane leak detection with methane leak sensors, the sensor placement techniques described herein are equally applicable to detection of any gaseous emission, whether methane or other gas, and whether a leak or other type of intentional or unintentional emission.

According to certain aspects, records are generated for candidate sensor locations based on wind rose data associated with a gas emission detection site. Candidate sensor locations may be selected for sensor placement using the record count for the candidate sensor locations. In some aspects, the sensor placement is based on mean record count. In some aspects, the candidate sensor locations may be ranked, for example in descending order, based on the mean record count. In some aspects, the candidate sensor location selection is sequentially performed based on the ranking. For example, a first candidate sensor location having the highest rank, or highest mean record count among the candidate sensor locations, may be selected for sensor placement. After a sensor location is selected, the sensor location is removed from the list of candidate sensor locations. A next sensor location may be selected from the list of the remaining candidate sensor locations, the next sensor location having the highest ranking, or highest mean record count, in the list of the remaining candidate sensor locations. The sensor locations may be performed sequentially until a desired number of sensors is reached (or based on another metric, such as a target coverage being reached). In some aspects, after a sensor location is selected, in addition to removing the selected sensor location from the list of candidate sensor locations, additional sensor locations within a specified minimum separation distance of the selected candidate sensor location may also be removed from the list of candidate sensor locations.

In some aspects, the deterministic and sequential sensor location selection using the record count may provide an automated and fast sensor location selection algorithm for gas emission sensor placement using reduced computational and time overhead, and may reduce human input and error.

The following description includes embodiments of the best mode presently contemplated for practicing the described implementations. This description is not to be taken in a limiting sense, but rather is made merely for the purpose of describing the general principles of the implementations. The scope of the described implementations should be ascertained with reference to the issued claims.

### Example Sequential Sensor Placement in Gas Emission Detection System Using Record Count

According to certain aspects, a site of interest for methane leak detection may be selected. In some aspects, the location of the selected site may be identified using a Global Positioning System (GPS) coordinates. In some aspects, the location of the selected site may be converted to a local reference system for evaluation purposes.

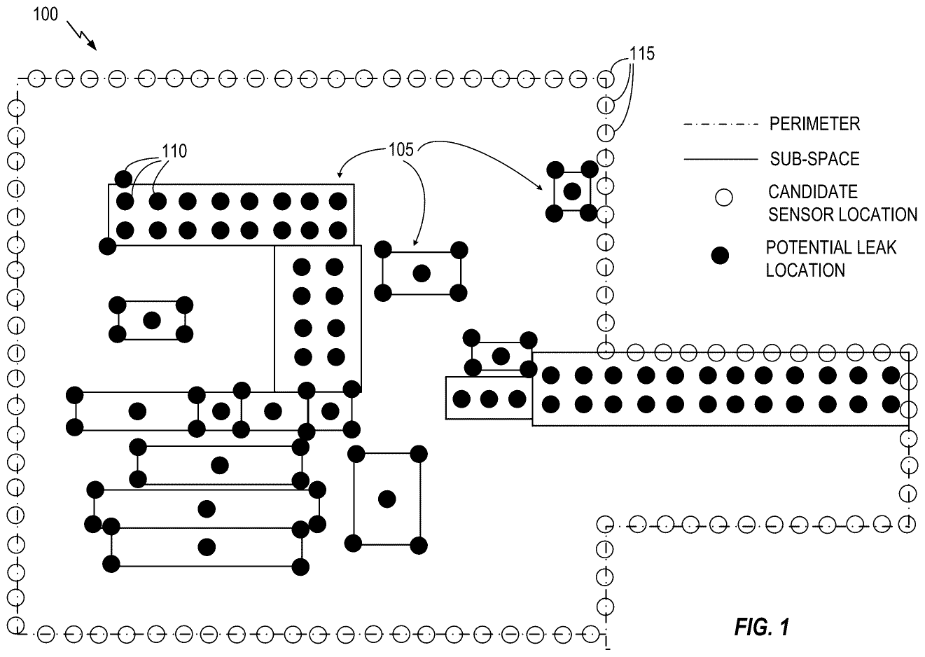

FIG. 1 depicts an example methane emission detection site 100. In some aspects, a perimeter of the methane emission detection site 100 may be defined (e.g., using a local coordinate system), as shown in FIG. 1.

In some aspects, sub-spaces 105 may be defined within the perimeter of the methane emission detection site 100. Sub-spaces 105 identify a region of interest for the methane emission detection. The sub-spaces 105 may restrict the area of the methane emission detection site 100 based on the locations of on-site equipment where potential methane leaks may occur. Thus, leak detection may be limited to points within the sub-spaces 105. As shown in FIG. 1, potential leak locations 110 may be specified within the sub-spaces 105. Some example methane leak candidates in oil and gas operations include, but are not limited to, wellheads, pipelines, compressors and pump stations, storage tanks, processing facilities, flares, valves and flanges, and pneumatic devices. Wellheads are the points at the surface where oil or gas wells are connected to production equipment. Leaks can occur due to faulty seals, equipment failure, or aging infrastructure. Pipelines span large areas. Methane can leak from pipelines due to corrosion, mechanical damage, or faulty joints. Compressors and pumping stations are used to maintain pressure and flow in the gas pipelines, and are prone to leaks, especially around seals, valves, and connections. Storage tanks are used to store natural gas or crude oil and can develop leaks, particularly at valves, hatches, and vents. Aging tanks and poor maintenance can exacerbate the risk of leaks. Processing facilities include gas processing plants, where gas is purified and separated from other components. Leaks can occur in valves, flanges, and processing equipment. Flares are used to burn off excess gas, flares can be leak candidates if they are not functioning correctly or if there is incomplete combustion, allowing methane to escape. Valves and flanges are common points of failure in all parts of the system; valves and flanges can develop leaks due to wear and tear, improper installation, or maintenance issues. Pneumatic devices are used to control equipment and can vent methane as part of their operation or due to malfunctions.

As shown in FIG. 1, candidate sensor locations 115 may be defined (e.g., specified or selected) within the methane emission detection site 100. In FIG. 1, the candidate sensor locations 115 are shown at the perimeter of the methane emission detection site 100, however, it should be understood that the candidate sensor locations 115 may be anywhere within the methane emission detection site 100. Further, while the candidate sensor locations 115 are shown in two dimensions (on the x- and y-planes) in FIG. 1, it should be understood that the candidate sensor locations 115 may be anywhere in three dimensions. That is, the candidate sensor locations 115 may additionally be defined at various heights or vertical locations (on the z-plane) within the methane emission detection site 100. In some aspects (not shown), restricted or exclusion zones may be defined within the methane emission detection site 100, where the restricted zones specify regions in which a sensor may not be placed (e.g., based on feasibility due to site restrictions). In some aspects, the candidate sensor locations 115 are typically outside of the sub-spaces 105.

According to certain aspects, a number of sensors for sensor placement optimization may be specified. For example, fewer sensors are placed than the total number of candidate sensor locations considered.

According to certain aspects, historical wind data may be obtained for the selected methane emission detection site 100. For example, existing historical wind data for a location nearest the intended methane emission detection site 100 may be obtained. For example, the GPS coordinates of the selected methane emission detection site 100 may be used to obtain the historical wind data for a location nearest to the GPS coordinates. In some aspects, the historical wind data may be obtained from a public database storing historical meteorological data.

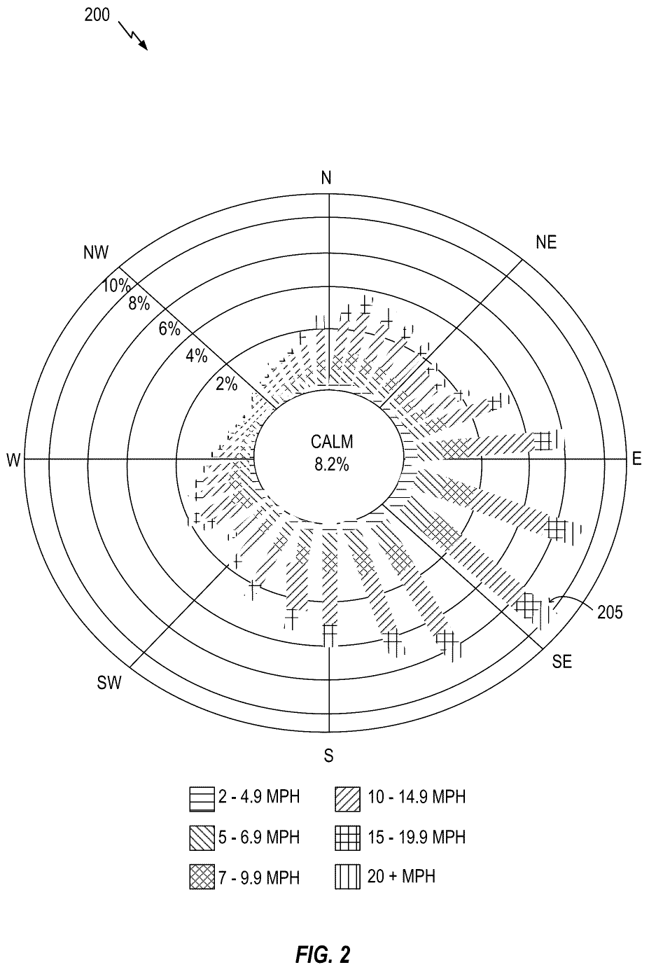

In some aspects, the historical wind data may be take the form of wind rose distributions. A wind rose distribution is a graphical tool used in meteorology to represent the distribution of wind speed and direction at a specific location over a certain period. A wind rose 200 may be presented as a radial plot, marked with the directions (e.g., north, east, south, west, etc.), as shown in FIG. 2. Each spoke or segment 205 on the wind rose represents a different direction from which the wind can blow. The length of each spoke indicates the frequency of winds coming from that direction, for example, a longer spoke means that wind from that direction was more common during the period. The spokes may be divided into segments, and each segment may be color-coded to represent different wind speed ranges. Thus, the spokes on the wind rose distribution can show not only how often the wind comes from a particular direction but also how strong the wind tends to be when it does. In some aspects, a wind rose may provide wind data collected over a long period (e.g., several years). For example, many airports have recorded historical wind data for decades.

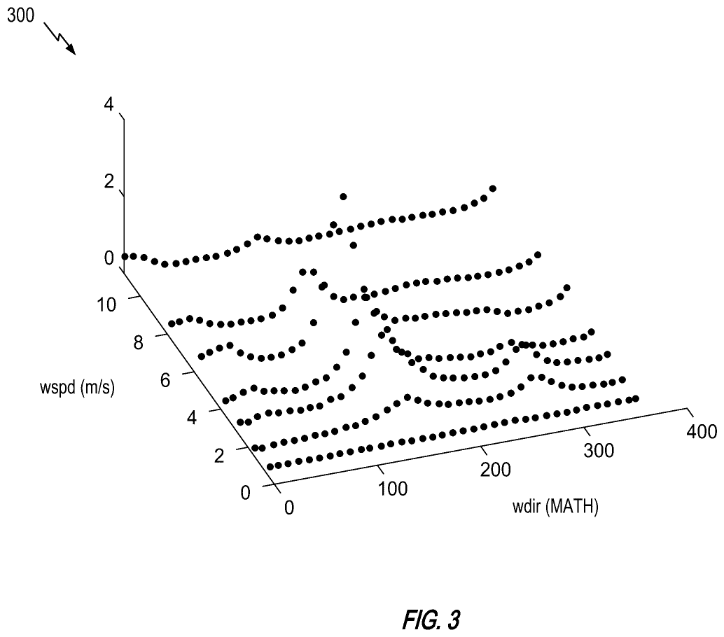

According to certain aspects, the wind rose 200 may be parsed to extract the underlying wind rose distribution data. From the wind rose distribution data, a probability of occurrence of a given wind direction and wind speed over the time period can be determined. FIG. 3 is a graph 300 illustrating an example wind rose distribution extracted from the wind rose 200. As shown, for example, a peak (i.e., with a high probability of occurrence) is noted at w_{dir}=135° and w_{spd}=6 m/s and a smaller peak (i.e., with a lower probability of occurrence) is noted at w_{dir}=280° and w_{spd}=2.5 m/s.

According to certain aspects, the window rose distribution data can be used to generate any number stochastic wind realizations. Thus, wind models based on historical data may be used for optimal sensor placement under wind uncertainty. Wind models serve as realizations of the wind field for sensor placement optimization with the aim of maximizing coverage for a given number of sensors. For example, a coverage metric may indicate the number or portion of candidate leaks that can be identified for a given sensor placement design under varying wind conditions. In some aspects, a wind realization represents the wind field as a function of time, say over 24 hours. Thus, the use of a greater number of wind realizations n_{w}, improves the sensor placement optimization by better accounting for the variability associated with the underlying wind field at the site. For example, in certain cases, a small number of wind realizations may be used (e.g., fewer than 10 wind realizations), however the use of a large number of wind realizations (e.g., in the thousands), say around 5000 realizations, the generated distribution will better match the actual distribution extracted from the wind rose. However, as discussed above, the use of a more wind realizations, will necessarily increase the computational overhead of the sensor placement optimization. Aspects of the present disclosure may permit efficient sensor placement design while using a large number of wind realizations.

According to certain aspects, the selected number of wind realizations n_{w}, the potential leak locations 110, and the candidate sensor locations 115 may be used to generate records. In some aspects, a leak (e.g., at a given rate) may be simulated at one of the potential leak locations and for one of the wind realizations and, using a model (e.g., such as a forward model, for example the GPM), predicted or simulated measurements of the leak can be obtained for a simulated or hypothetical sensor at each of the candidate sensor locations to obtain the simulated records for the candidate sensor locations, where each record is associated with the methane concentration reading, wind speed, and wind direction. This can be repeated at the potential leak location for each of the plurality of wind realizations. The leak simulations subject to the plurality of wind realizations can be further repeated for leaks at each of the plurality of potential leak locations. Thus, records can be generated for each of the candidate sensor locations for sensor measurements of every combination of the wind realizations and potential leak locations. In some aspects, for each candidate sensor locations 115, records are generated over each of the selected wind realizations and for each potential leak location 110. For example, a methane plume may be modeled at a potential leak location 110, and a given wind realization (w_{dir}, w_{spd}) may be modeled to act on the methane plume. For the methane plume at the potential leak location 110 under the wind realization, methane concentration measurements can be estimated (e.g., simulated) over time at a candidate sensor location 115 to generate records. A record may be “counted” when the methane concentration detected is at a sufficiently high level (e.g., a specified threshold, or a specified threshold above a background methane concentration level). The record generation can be repeated over each combination of the selected n_{w} wind realizations and nₑ potential leak locations 110, and for all of the candidate sensor locations 115.

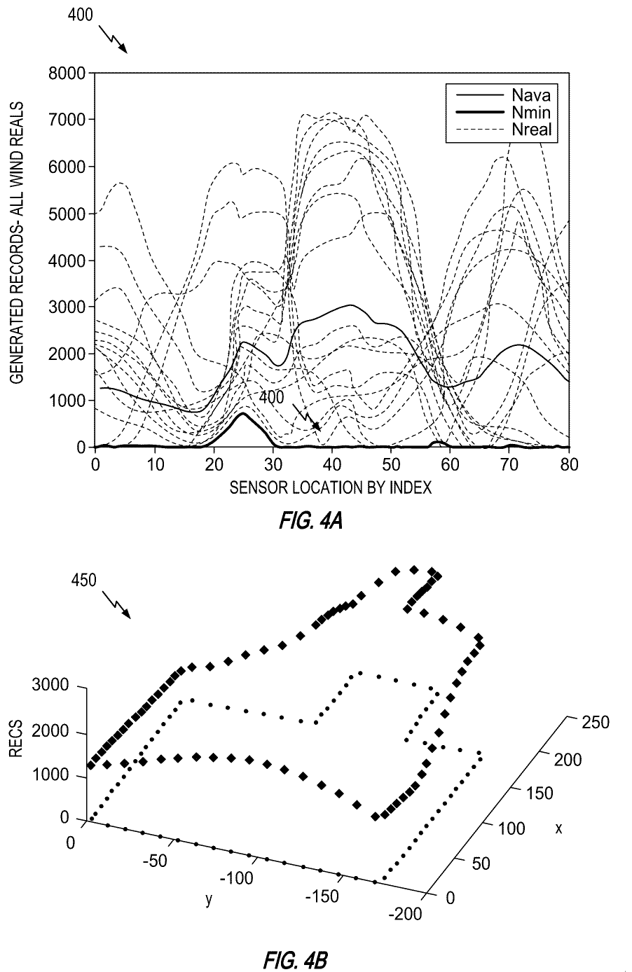

According to certain aspects, for each candidate sensor location 115, the number of realizations generated over the nₑ potential leak locations 110 and the selected n_{w} wind realizations is totaled to determine a mean number of records generated at each candidate sensor location 115. In some aspects, the mean record count at each candidate sensor location 115 may be determined by processing each potential leak location 110 over each wind realization, and taking average number of records generated over all wind realizations at each given candidate sensor location 115. In some aspects, the record count per candidate sensor location 115 for all selected wind realizations can be plotted along with the mean record count per candidate sensor location 115, as shown in the graph 400 in FIG. 4A. In some aspects, the mean record count for each candidate sensor location 115 can be plotted at the pre-defined candidate sensor locations 115 as shown in the graph 450 in FIG. 4B. It should be understood that while FIGS. 4A-4B illustrate record generation using 25 wind realizations, techniques described herein may be used to generate records using any desired number of wind realizations.

According to certain aspects, once all of the candidate sensor locations 115 have been evaluated (over the n_{w} realizations and the nₑ leak points), the candidate sensor locations 115 can be ranked based on the mean record count established. In some aspects, the candidate sensor locations 115 are ranked in order highest mean record count to lowest mean record count.

According to certain aspects, a number sensors, nₛ, to place at the methane emission detection site 100 may be specified, selected, or pre-defined. In some aspects, after generating the records and ranking the mean record count for the candidate sensor locations 115, the mean record counts may be used for placement selection of the nₛ sensors at nₛ of the candidate sensor locations 115.

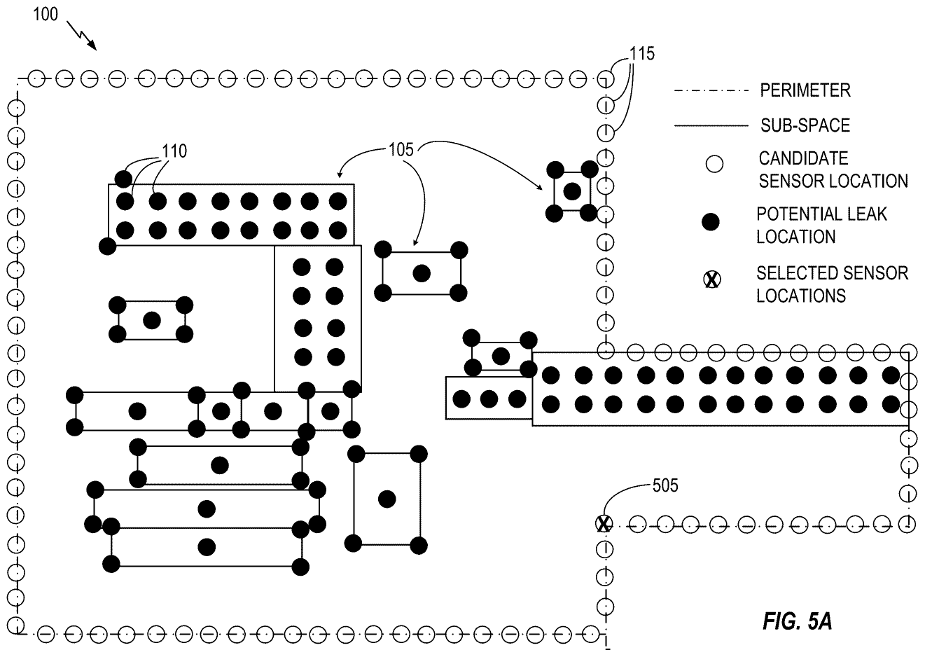

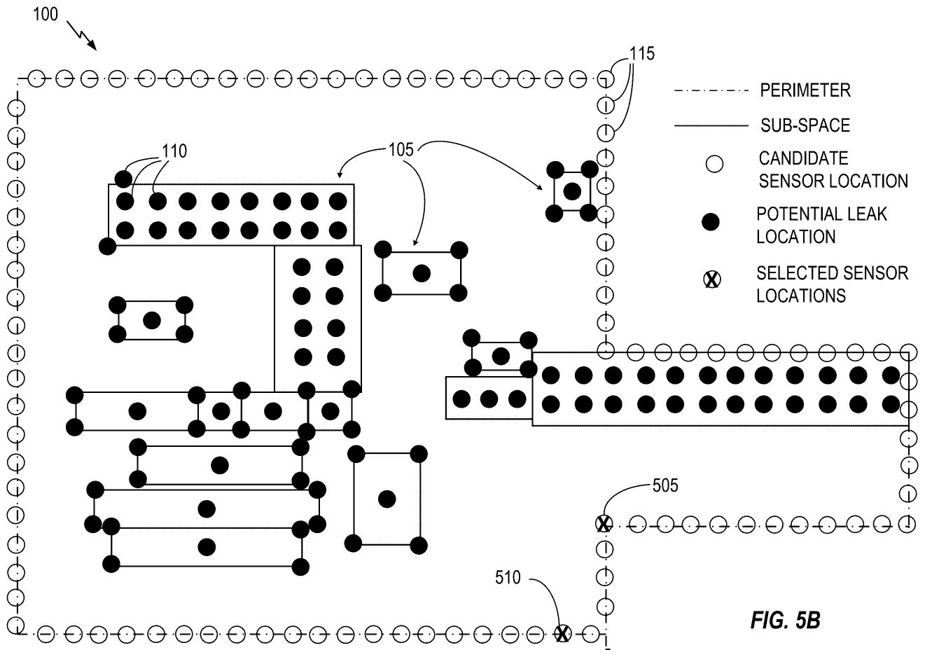

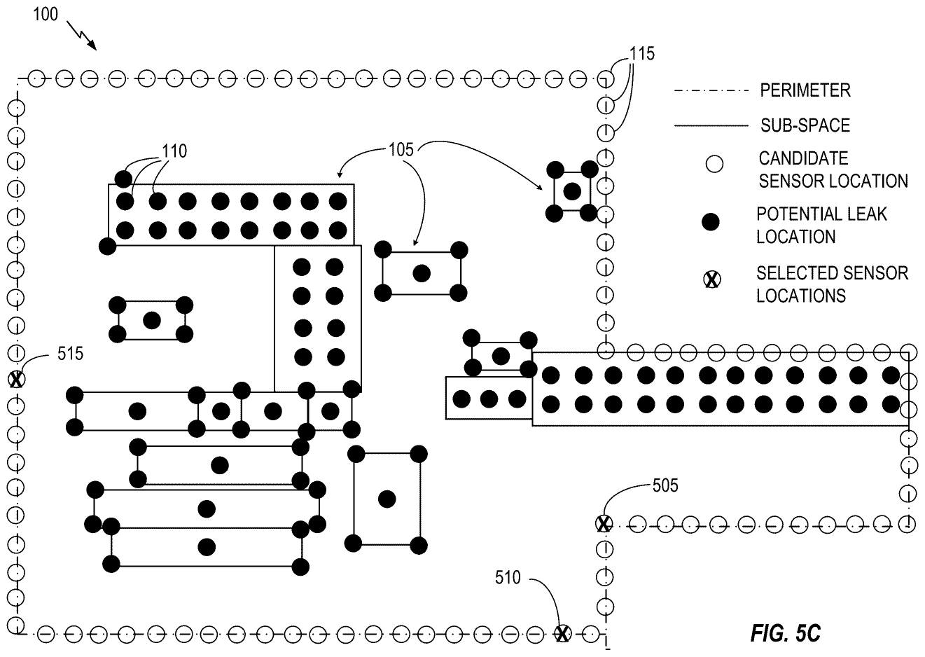

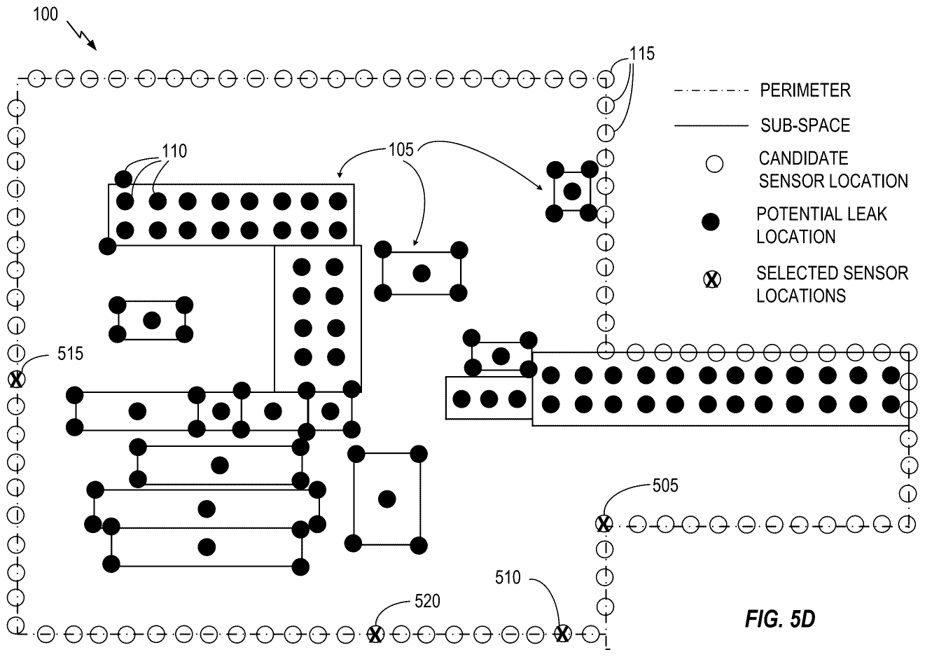

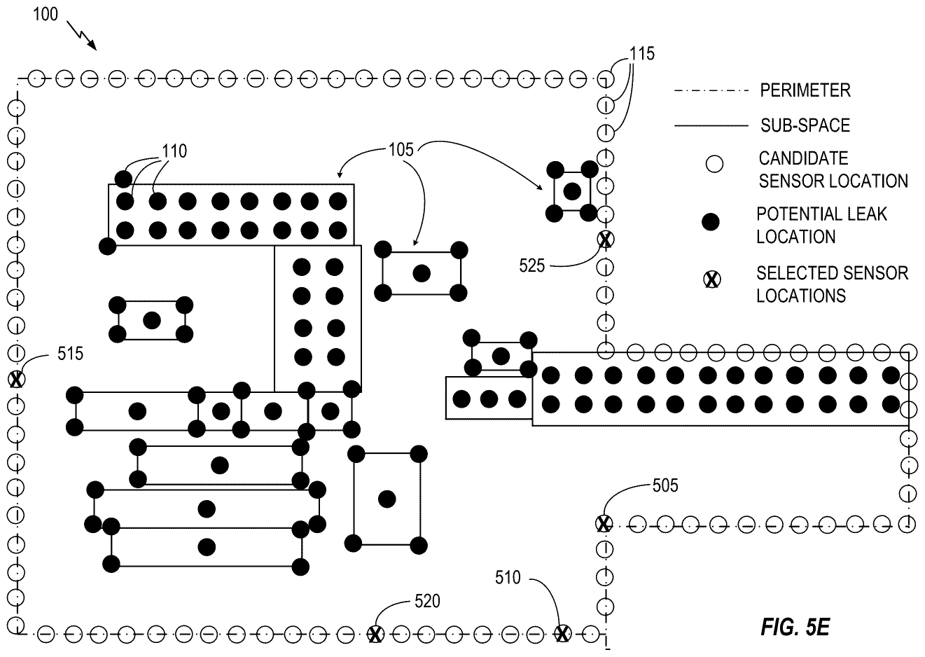

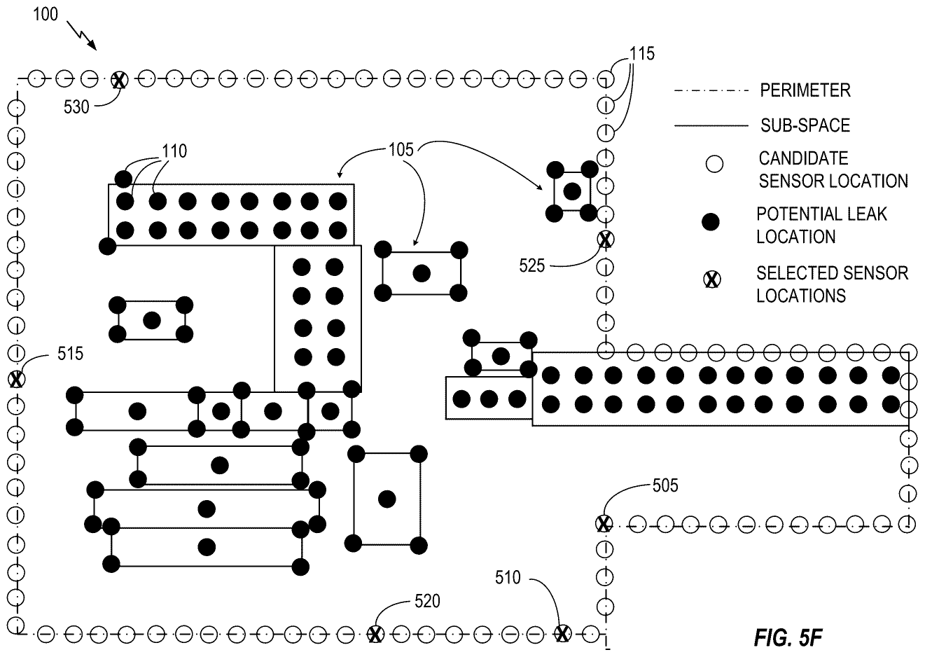

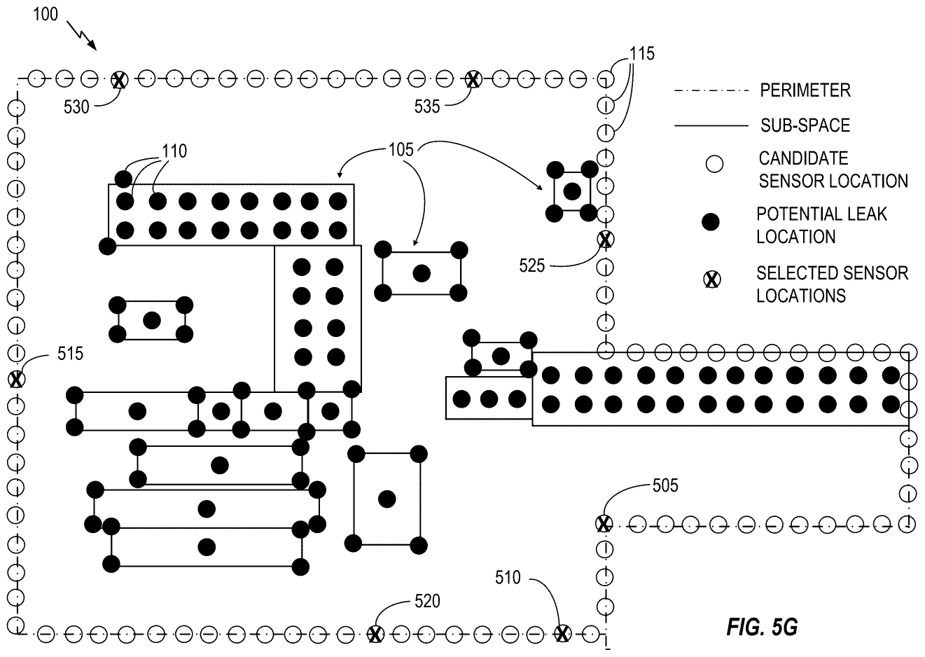

FIGS. 5A-5G illustrate example sensor placement selection for the methane emission detection site 100 for nₛ=7 sensors. It should be understood that while FIGS. 5A-5G illustrate candidate sensor location selection and sensor placement of 7 sevens, techniques described herein may be used to select any desired number of sensors from any number of candidate sensor locations. In some aspects, the sensor placement selection is automatic and sequential.

According to certain aspects, the candidate sensor location 505 with the highest rank (e.g., the highest mean record count) may be selected as the first sensor placement location as shown in FIG. 5A. That candidate sensor location 505 is then removed from the list of candidate sensor locations 115.

According to certain aspects, after removing the selected candidate sensor location 505 from the list, the candidate sensor location 510 with the highest rank is then selected from the remaining candidates in the list of candidate sensor locations 115 as shown in FIG. 5B, and the selected candidate sensor location 510 is then removed from the list of candidates. This sequential selection may continue until the nₛ candidate sensor locations 515, 520, 525, 530, 535 are selected as shown in FIGS. 5C-5G.

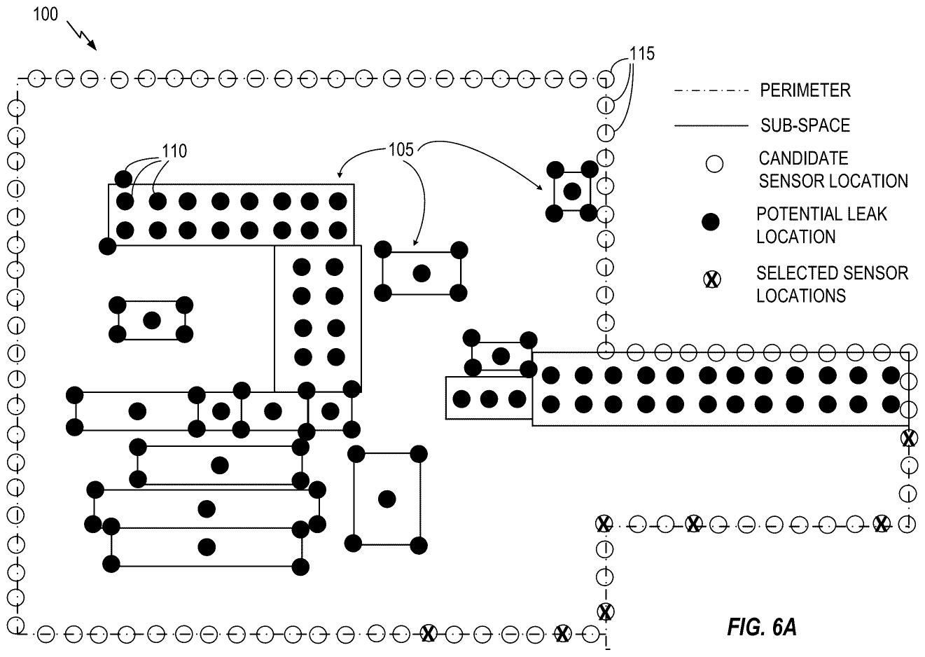

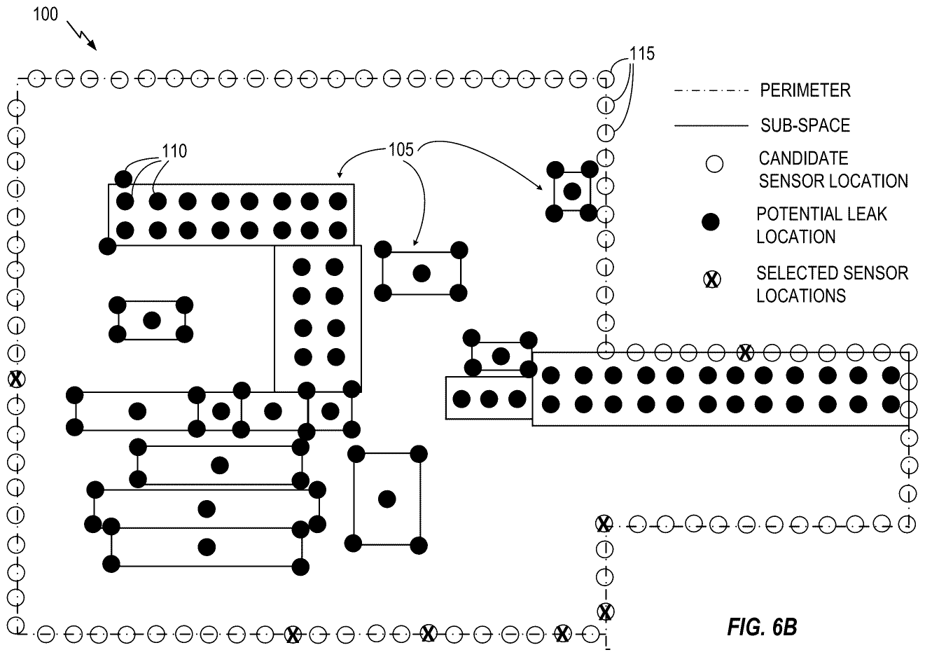

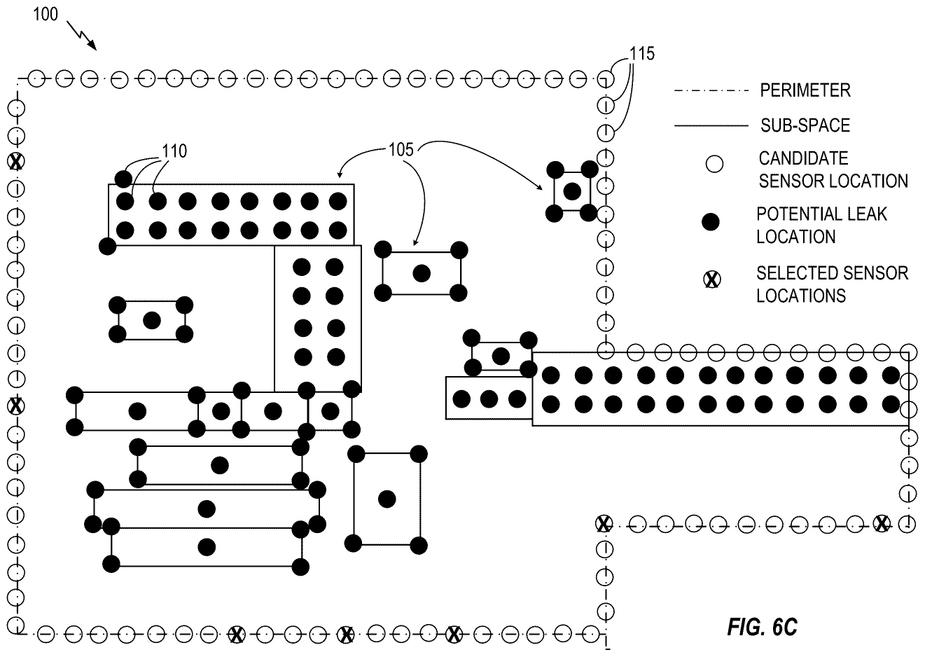

In some aspects, after the selection of a candidate sensor location 115, in addition to removing the selected candidate sensor location from the list of candidates, all other candidate sensor locations that lie within a specified physical separation distance from the selected candidate sensor location 115 are also removed from the list of candidates before the selection of the next candidate sensor location from the remaining candidate sensor locations in the list. The use of a separation distance prevents the placement of sensors at candidate sensor locations that are close together, which may be redundant, and allows better coverage from the sensors. FIGS. 5G and 6A-6C illustrate example sensor placement selections using different separation distances. For example, FIG. 5G may be associated with a largest separation distance. As shown, the larger separation distance may result in a relatively even distribution of the sensor placements among the candidate sensor locations 115. FIG. 6A may be associated with a small, or no, separation distance. As shown, little to no separation distance may result in a relatively tightly clustered distribution of the sensor placements among the candidate sensor locations 115. FIG. 6B may be associated with a next separation distance, larger than the separation distance of FIG. 6A but smaller than the separation distance of FIG. 6C. FIG. 6C may be associated with a next separation distance, larger than the separation distance of FIG. 6B but smaller than the separation distance of FIG. 5G. As shown, as separation distance increases, the sensor placements among the candidate sensor locations 115 may become more evenly distributed.

According to certain aspects, the sequential sensor placement using the mean record count may eliminate or simplify a design evaluation process. In some aspects, the sequential sensor placement using the mean record eliminates the computations of inversions associated with optimal candidate sensor placement, thereby reducing the overhead and making the selection process fast and automatic

According to certain aspects, other metrics associated with the record count of the candidate sensor locations 115 may be used for the sensor placement selection. In some aspects, a minimum or maximum record count may be used to rank the candidate sensor locations 115 for sensor placement selection.

According to certain aspects, after selecting the nₛ sensor locations, sensors may be installed at the selected locations.

According to certain aspects, after installation of the nₛ sensor, the sensors may be used to monitor the methane emission detection site 100 for concentrations of methane. In some aspects, the sensors are configured to perform continuous measurement. In some aspects, the sensors perform methane concentration measurements. In some aspects, a sensor may also measure wind data (if provisioned with an anemometer). The sensors are used to generate records based on the data collected at the sensor location.

According to certain aspects, the measurements (noted as records) are processed to detect leak or emission locations. In some aspects, inversions are performed using the records to detect the leak location. For example, a Gaussian plume model may be used to quantify the leak source using the measurements from the sensors at known locations along with the prevailing wind conditions (as given by the generated records).

According to certain aspects, when a leak or emission is detected, steps may be taken to mitigate or eliminate the leak or emission. In some aspects, operations of the equipment at the leak or emission location may be paused. In some aspects, engineers or workers may be deployed to the leak or emission location in order to replace or repair the equipment.

The sequential sensor placement selection using record count techniques described may decrease the time required for sensor placement selection. For example, a sensor placement selection with 200 candidate sensor locations, 25 wind realizations, and 116 potential leak locations may be performed in less than hour, in contrast to the 3.5 days of time to perform a conventional sensor placement optimization for only 5 wind realizations and the 116 potential leak locations. In addition, because many more wind realizations can be used to generate the records, the sensor placement design (indicative of coverage estimation) may be more meaningful as the actual wind rose distribution will be more representative. Further, the sensor placement selection technique described is quantitative and deterministic. Accordingly, the sensor placement selection process may be automatic, which may reduce or eliminate the need for human input in the sensor placement selection process.

### Example Operations for Sensor Placement for Gas Emission Detection Using Record Count

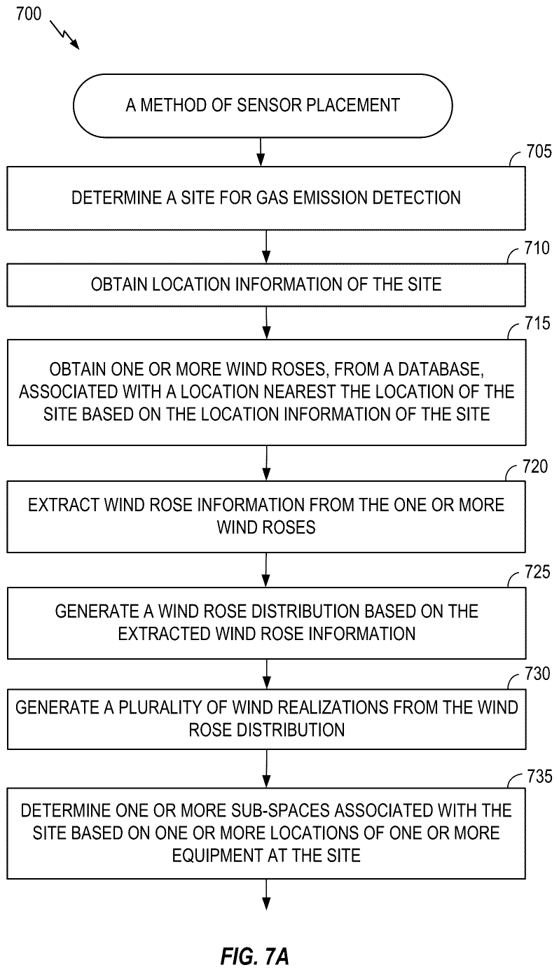

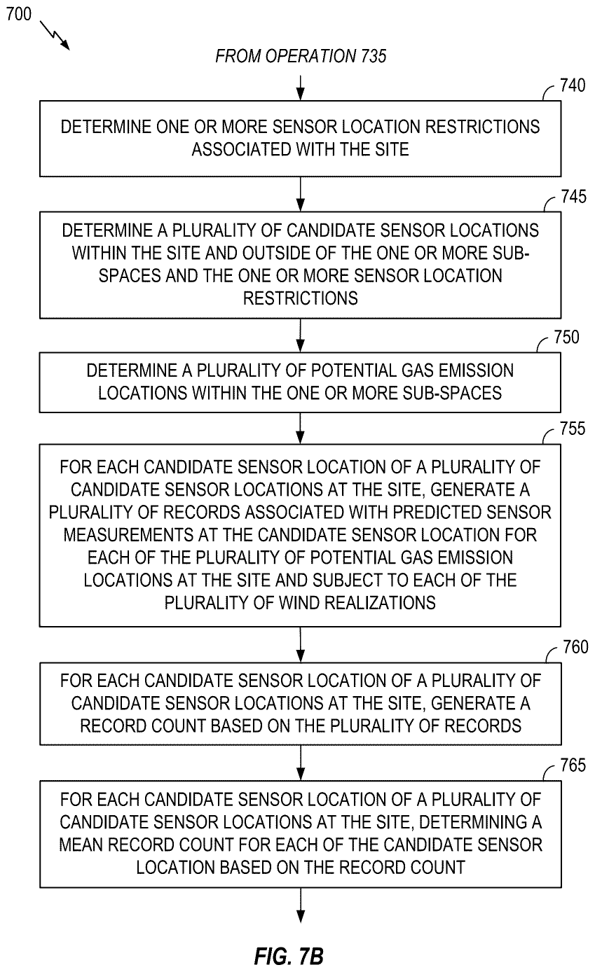

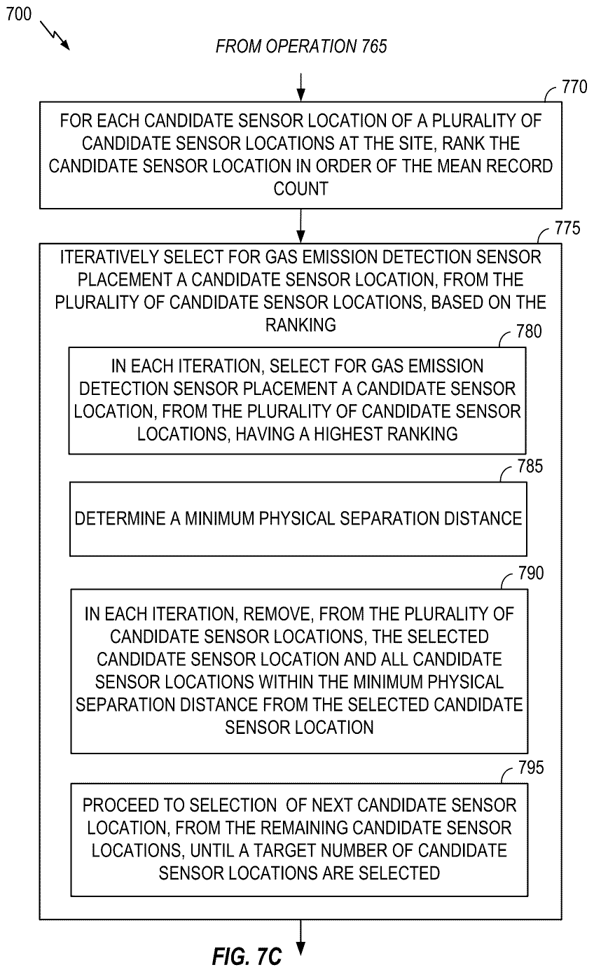

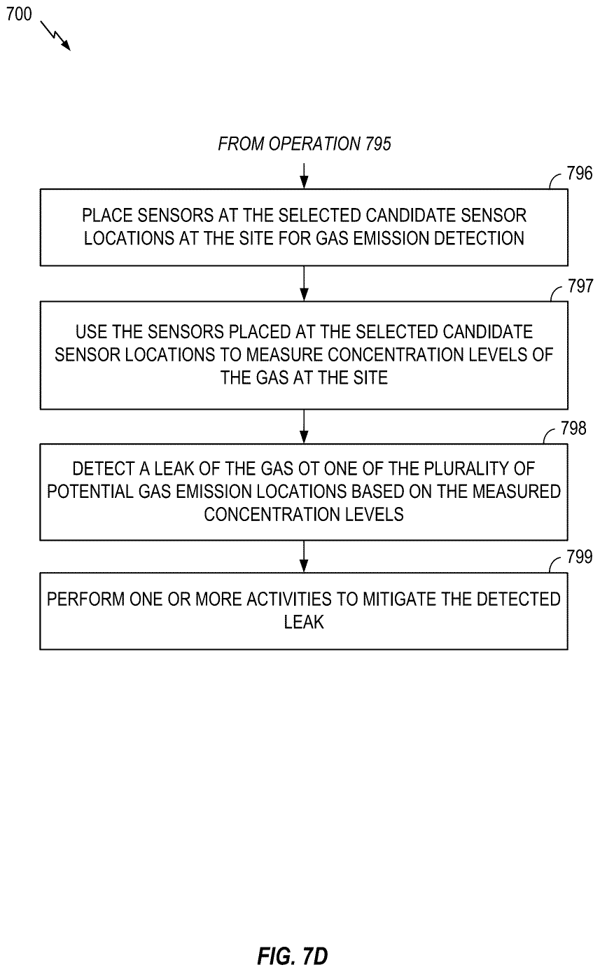

FIGS. 7A-7D depict a flow diagram depicting an example operations 700 for sensor placement. In some aspects, aspects of the operations 700 may be performed by a gas emission detection system, which may include local, remote, cloud, virtualized, and/or distributed hardware components.

As shown in FIG. 7A, the operations 700 may include, optionally, at operation 705, determining the site for gas emission detection.

The operations 700 may include, optionally, at operation 710, obtaining location information of the site.

The operations 700 may include obtaining wind rose distribution data associated with the site. The operations 700 may include, optionally, at operation 715, obtaining one or more wind roses, from a database, associated with a location nearest the location of the site based on the location information of the site. The operations 700 may include, optionally, at operation 720, extracting wind rose information from the one or more wind roses. The operations 700 may include, optionally, at operation 725, generating a wind rose distribution based on the extracted wind rose information.

The operations 700 may include at operation 730, generating a plurality of wind realizations from the wind rose distribution data.

The operations 700 may include determining a plurality of candidate sensor locations. The operations 700 may include, optionally, at operation 735, determining one or more sub-spaces associated with the site based on one or more locations of one or more equipment at the site. As shown in FIG. 7B, the operations 700 may include, optionally, at operation 740, determining one or more sensor location restrictions associated with the site. The operations 700 may include, optionally, at operation 745, determining the plurality of candidate sensor locations within the site, outside of the one or more sub-spaces, and excluding the one or more sensor location restrictions. In some aspects, one or more of plurality of the candidate sensor locations is located within a perimeter of the site. In some aspects, the plurality of candidate sensor locations comprise candidate sensor locations at different heights.

The operations 700 may include, optionally, at operation 750, determining a plurality of potential gas emission locations within the one or more sub-spaces.

The operations 700 may include, at operation 755, for each candidate sensor location of a plurality of candidate sensor locations at the site, generating a plurality of records associated with predicted sensor measurements at the candidate sensor location for each of the plurality of potential gas emission locations at the site and subject to each of the plurality of wind realizations.

The operations 700 may include, at operation 760, for each candidate sensor location of a plurality of candidate sensor locations at the site, generating a record count based on the plurality of records.

The operations 700 may include for each candidate sensor location of a plurality of candidate sensor locations at the site, ranking the candidate sensor location based on the record count. The operations 700 may include, at operation 765, for each candidate sensor location of a plurality of candidate sensor locations at the site, determining a mean record count for each of the candidate sensor location based on the record count. As shown in FIG. 7C, the operations 700 may include, at operation 770, for each candidate sensor location of a plurality of candidate sensor locations at the site, ranking the candidate sensor location order of the mean record count. In some aspects, the system automatically ranks the candidate sensor locations based on the record count.

The operations 700 may include, at operation 775, iteratively selecting candidate sensor locations, from the plurality of candidate sensor locations, based on the ranking. The iteratively selecting at operation 775 includes, in each iteration, at operation 780, selecting for gas emission detection sensor placement a candidate sensor location, from the plurality of candidate sensor locations, having a highest ranking. The operations 700 may include, optionally, at operation 785, determining a minimum physical separation distance. The iteratively selecting at operation 775 includes, in each iteration, at operation 790, removing, from the plurality of candidate sensor locations, the selected candidate sensor location and all candidate sensor locations within the minimum physical separation distance from the selected candidate sensor location. In some aspects, the system automatically iteratively selects candidate sensor locations, from the plurality of candidate sensor locations, based on the ranking. The operations 700 may include, optionally, at operation 795, proceeding to the selection of a next candidate sensor location, from the remaining candidate sensor locations, until a target number of candidate sensor locations are selected.

As shown in FIG. 7D, the operations 700 may include, at operation 796, placing sensors at the selected candidate sensor locations at the site for gas emission detection.

The operations 700 may include, optionally, at operation 797, using the sensors placed at the selected candidate sensor locations to measure concentration levels of the gas at the site.

The operations 700 may include, optionally, at operation 798, detecting a leak of the gas at one of the plurality of potential gas emission locations based on the measured concentration levels.

The operations 700 may include, optionally, at operation 799, performing one or more activities to mitigate the detected leak.

### Example System for Gas Emission Detection Sensor Placement Selection Using Record Count

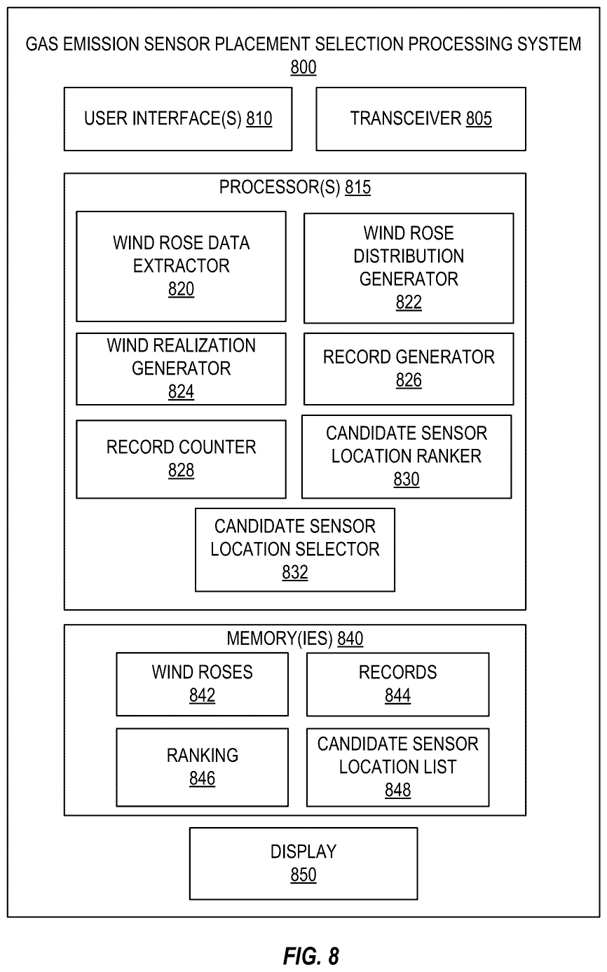

According to certain aspects, a gas emission detection sensor placement selection processing system 800 is provided, as shown in FIG. 8. The gas emission detection sensor placement selection system 800 may run on a single computing device or across multiple devices.

As shown, the gas emission detection sensor placement selection system 800 may include one or more user interfaces 810 on the one or more devices comprising the gas emission detection sensor placement selection system 800 that allow a user to interact with the gas emission detection sensor placement selection system 800. In some examples, the user interface(s) 810 may include a graphical user interface (GUI) that display to a user and/or accepts touch screen inputs from the user. The user interface(s) 810 may include one or more input/output (IOs) interfaces that allows one or more I/O devices (e.g., keyboards, displays, mouse devices, pen inputs, microphones, etc.) to connect to the gas emission detection sensor placement selection system 800. In some aspects, the user interface(s) 810 are configured to receive user input, including one or more of site selection, site location information, candidate sensor location, sub-space indication, sensor placement restriction, number of candidate sensor locations, number of sensors for placement, and number of wind realizations as described herein.

As shown in FIG. 8, the gas emission detection sensor placement selection system 800 may include a transceiver 805 and one or more network interface(s). The transceiver 805 and network interface(s) may allow the gas emission detection sensor placement selection system 800 to connect to a network (e.g., such as the Internet, a local area network (LAN), a wireless LAN (WLAN), a wireless wide area network (WWAN), Wi-Fi, etc.) and/or to communicate with other devices, such as to communicatively connect devices within the gas emission detection sensor placement selection system 800 and/or devices external to the gas emission detection sensor placement selection system 800. In some aspects, the transceiver 805 and one or more network interface(s) may be configured to receive one or more of wind rose, wind rose data, wind rose distribution, and sensor measurement data as described herein.

As shown, the gas emission detection sensor placement selection system 800 may include a processing system including one or more processor(s) 815. The one or more processor(s) 815 may be local to the gas emission detection sensor placement selection system 800 or remote (e.g., cloud computing resources). The one or more processor(s) 815 may comprise one or more central processing units (CPUs). The CPU may have multiple processing cores. In some aspects, the processor(s) 815 may include a wind rose data extractor processor 820 configured to extract wind rose data from one or more stored wind roses 842 as described herein. In some aspects, the processor(s) 815 may include a wind rose distribution generator processor 822 configured to generate a wind rose distribution from the extracted wind rose data as described herein. In some aspects, the processor(s) 815 may include a wind realization generator processor 824 configured to generate a plurality of wind realizations from the wind rose distribution as described herein. In some aspects, the processor(s) 815 may include a record generator processor 826 configured to generate records for each candidate sensor location using the plurality of wind realizations as described herein. In some aspects, the processor(s) 815 may include a record counter processor 828 configured to count the records generated for each of the candidate sensor locations as described herein. In some aspects, the processor(s) 815 may include a candidate sensor location ranker processor 830 configured to rank the candidate sensor locations based on the record count as described herein. In some aspects, the processor(s) 815 may include a candidate sensor location selector processor 832 configured to sequentially select candidate sensor locations for sensor placement based on the ranking as described herein.

The processing system may further include memory(ies) 840 and/or storage(s), which may be local to the gas emission detection sensor placement selection system 800 or remote (e.g., cloud storage). The memory 840 may represent a random access memory (RAM). The storage may be a disk drive, a combination of fixed or removable storage devices, such as fixed disc drives, removable memory cards or optical storage, network attached storage (NAS), or a storage area-network (SAN). The CPU may retrieve and execute programming instructions stored in the memory(ies) 840. Similarly, the CPU may retrieve and store application data residing in the memory(ies) 840. As shown in FIG. 8, the memory(ies) 840 may store one or more of wind roses 842 associated with the gas emission detection site, records 844 generated for the candidate sensor locations, rankings 846 of the candidate sensor locations, and candidate sensor location list 848 as described herein.

### Example Clauses

Implementation examples are described in the following numbered aspects:

Aspect 1: A method for sensor placement, the method comprising: obtaining wind rose distribution data associated with a site for gas emission detection; generating a plurality of wind realizations from the wind rose distribution data; for each candidate sensor location of a plurality of candidate sensor locations at the site: generating a plurality of records associated with predicted sensor measurements at the candidate sensor location for each of a plurality of potential gas emission locations at the site and subject to each of the plurality of wind realizations; generating a record count based on the plurality of records; and ranking the candidate sensor location based on the record count; iteratively selecting candidate sensor locations, from the plurality of candidate sensor locations, based on the ranking, wherein the iteratively selecting comprises, in each iteration: selecting for gas emission detection sensor placement a candidate sensor location, from the plurality of candidate sensor locations, having a highest ranking; and removing at least the selected candidate sensor location from the plurality of candidate sensor locations; and placing sensors at the selected candidate sensor locations at the site for gas emission detection.

Aspect 2: The method of Aspect 1, further comprising: determining the site for gas emission detection; obtaining location information of the site; and obtaining the wind rose distribution data based on the location information of the site.

Aspect 3: The method of Aspect 2, wherein obtaining the wind rose distribution data based on the location information of the site comprises: obtaining one or more wind roses, from a database, associated with a location nearest the location of the site; extracting wind rose information from the one or more wind roses; and generating a wind rose distribution based on the extracted wind rose information.

Aspect 4: The method of any combination of Aspects 1-3, further comprising: determining one or more sub-spaces associated with the site based on one or more locations of one or more equipment at the site; determining the plurality of potential gas emission locations within the one or more sub-spaces; and determining the plurality of candidate sensor locations within the site and outside the one or more sub-spaces.

Aspect 5: The method of any combination of Aspects 1-4, further comprising: determining one or more sensor location restrictions associated with the site; and determining the plurality of candidate sensor locations excluding the one or more sensor location restrictions.

Aspect 6: The method of any combination of Aspects 1-5, further comprising determining a mean record count for each of the plurality of candidate sensor locations based on the record count, wherein ranking the candidate sensor location based on the record count comprises ranking the candidate sensor location in descending order of mean record count.

Aspect 7: The method of any combination of Aspects 1-6, further comprising determining a minimum physical separation distance, wherein removing at least the selected candidate sensor location from the plurality of candidate sensor locations comprises further removing, from the plurality of candidate sensor locations, all candidate sensor locations within the minimum physical separation distance from the selected candidate sensor location.

Aspect 8: The method of any combination of Aspects 1-7, wherein the ranking the candidate sensor locations based on the record count and the iteratively selecting candidate sensor locations, from the plurality of candidate sensor locations, based on the ranking comprises: automatically ranking the candidate sensor locations based on the record count; and automatically iteratively selecting candidate sensor locations, from the plurality of candidate sensor locations, based on the ranking.

Aspect 9: The method of any combination of Aspects 1-8, further comprising: using the sensors at the selected candidate sensor locations to measure concentrations levels of the gas at the site; detecting a leak of the gas at one of the plurality of potential gas emission locations based on the measured concentration levels; and performing one or more activities to mitigate the leak.

Aspect 10: The method of any combination of Aspects 1-9, wherein: one or more of plurality of the candidate sensor locations is located within a perimeter of the site; and the plurality of candidate sensor locations comprise candidate sensor locations at different heights.

Aspect 11: An apparatus for performing the candidate sensor location selection of any combination of aspects 1-10.

Aspect 12: A non-transitory computer readable medium for performing the candidate sensor location selection of any combination of aspects 1-10.

### Additional Considerations

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the general principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from that described, and various actions may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

The various illustrative logical blocks, modules and circuits described in connection with the present disclosure may be implemented or performed with a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, a system on a chip (SoC), or any other such configuration.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more actions for achieving the methods. The method actions may be interchanged with one another without departing from the scope of the claims. In other words, unless a specific order of actions is specified, the order and/or use of specific actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for”. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for sensor placement, the method comprising:
obtaining wind rose distribution data associated with a site for gas emission detection;
generating a plurality of wind realizations from the wind rose distribution data;
for each candidate sensor location of a plurality of candidate sensor locations at the site:
generating a plurality of records associated with predicted sensor measurements at the candidate sensor location for each of a plurality of potential gas emission locations at the site and subject to each of the plurality of wind realizations;
generating a record count based on the plurality of records; and
ranking the candidate sensor location based on the record count;
iteratively selecting candidate sensor locations, from the plurality of candidate sensor locations, based on the ranking, wherein the iteratively selecting comprises, in each iteration:
selecting for gas emission detection sensor placement a candidate sensor location, from the plurality of candidate sensor locations, having a highest ranking; and
removing at least the selected candidate sensor location from the plurality of candidate sensor locations; and
placing sensors at the selected candidate sensor locations at the site for gas emission detection.

2. The method of claim 1, further comprising:
determining the site for gas emission detection;
obtaining location information of the site; and
obtaining the wind rose distribution data based on the location information of the site.

3. The method of claim 2, wherein obtaining the wind rose distribution data based on the location information of the site comprises:
obtaining one or more wind roses, from a database, associated with a location nearest the location of the site;
extracting wind rose information from the one or more wind roses; and
generating a wind rose distribution based on the extracted wind rose information.

4. The method of claim 1, further comprising:
determining one or more sub-spaces associated with the site based on one or more locations of one or more equipment at the site;
determining the plurality of potential gas emission locations within the one or more sub-spaces; and
determining the plurality of candidate sensor locations within the site and outside the one or more sub-spaces.

5. The method of claim 1, further comprising:
determining one or more sensor location restrictions associated with the site; and
determining the plurality of candidate sensor locations excluding the one or more sensor location restrictions.

6. The method of claim 1, further comprising determining a mean record count for each of the plurality of candidate sensor locations based on the record count, wherein ranking the candidate sensor location based on the record count comprises ranking the candidate sensor location in descending order of mean record count.

7. The method of claim 1, further comprising determining a minimum physical separation distance, wherein removing at least the selected candidate sensor location from the plurality of candidate sensor locations comprises further removing, from the plurality of candidate sensor locations, all candidate sensor locations within the minimum physical separation distance from the selected candidate sensor location.

8. The method of claim 1, wherein the ranking the candidate sensor locations based on the record count and the iteratively selecting candidate sensor locations, from the plurality of candidate sensor locations, based on the ranking comprises:
automatically ranking the candidate sensor locations based on the record count; and
automatically iteratively selecting candidate sensor locations, from the plurality of candidate sensor locations, based on the ranking.

9. The method of claim 1, further comprising:
using the sensors placed at the selected candidate sensor locations to measure concentration levels of the gas at the site;
detecting a leak of the gas at one of the plurality of potential gas emission locations based on the measured concentration levels; and
performing one or more activities to mitigate the leak.

10. The method of claim 1, wherein:
one or more of plurality of the candidate sensor locations is located within a perimeter of the site; and
the plurality of candidate sensor locations comprise candidate sensor locations at different heights.

11. A computer readable medium storing computer executable code for automated sensor placement location selection, the computer executable code comprising:
code for obtaining wind rose distribution data associated with a site for gas emission detection;
code for generating a plurality of wind realizations from the wind rose distribution data;
code for, for each candidate sensor location of a plurality of candidate sensor locations at the site, generating a plurality of records associated with predicted sensor measurements at the candidate sensor location for each of a plurality of potential gas emission locations at the site and subject to each of the plurality of wind realizations;
code for, for each candidate sensor location of a plurality of candidate sensor locations at the site, generating a record count based on the plurality of records;
code for, for each candidate sensor location of a plurality of candidate sensor locations at the site, ranking the candidate sensor location based on the record count;
code for iteratively selecting candidate sensor locations, from the plurality of candidate sensor locations, based on the ranking, wherein the code for iteratively selecting comprises:
code for, in each iteration, selecting for gas emission detection sensor placement a candidate sensor location, from the plurality of candidate sensor locations, having a highest ranking; and
code for, in each iteration, removing at least the selected candidate sensor location from the plurality of candidate sensor locations; and
code for outputting the selected sensor locations.

12. The computer readable medium of claim 11, the computer executable code further comprising:
code for determining the site for gas emission detection;
code for obtaining location information of the site; and
code for obtaining the wind rose distribution data based on the location information of the site.

13. The computer readable medium of claim 11, wherein the code for obtaining the wind rose data based on the location information of the site comprises:
code for obtaining one or more wind roses, from a database, associated with a location nearest the location of the site;
code for extracting wind rose information from the one or more wind roses; and
code for generating a wind rose distribution based on the extracted wind rose information.

14. The computer readable medium of claim 11, the computer executable code further comprising:
code for determining one or more sub-spaces associated with the site based on one or more locations of one or more equipment at the site;
code for determining the plurality of potential gas emission locations within the one or more sub-spaces; and
code for determining the plurality of candidate sensor locations within the site and outside the one or more sub-spaces.

15. The computer readable medium of claim 11, the computer executable code further comprising:
code for determining one or more sensor location restrictions associated with the site; and
code for determining the plurality of candidate sensor locations excluding the one or more sensor location restrictions.

16. The computer readable medium of claim 11, the computer executable code further comprising code for determining a mean record count for each of the plurality of candidate sensor locations based on the record count, wherein the code for ranking the candidate sensor location based on the record count comprises code for ranking the candidate sensor location in descending order of mean record count.

17. The computer readable medium of claim 11, the computer executable code further comprising code for determining a minimum physical separation distance, wherein the code for removing at least the selected candidate sensor location from the plurality of candidate sensor locations comprises code for further removing, from the plurality of candidate sensor locations, all candidate sensor locations within the minimum physical separation distance from the selected candidate sensor location.

18. The computer readable medium of claim 11, the computer executable code further comprising:
code for obtaining measured concentrations levels of the gas at the site from the sensors at the selected candidate sensor locations;
code for detecting a leak of the gas at one of the plurality of potential gas emission locations based on the measured concentration levels; and
code for performing one or more activities to mitigate the leak.

19. An apparatus for automated sensor placement location selection, the apparatus comprising:
one or more memories storing computer executable code; and
one or more processors configured to execute the computer executable code and cause the apparatus to:
obtain wind rose distribution data associated with a site for gas emission detection;
generate a plurality of wind realizations from the wind rose distribution data; for each candidate sensor location of a plurality of candidate sensor locations at the site:
generate a plurality of records associated with predicted sensor measurements at the candidate sensor location for each of a plurality of potential gas emission locations at the site and subject to each of the plurality of wind realizations;
generate a record count based on the plurality of records; and
rank the candidate sensor location based on the record count;
iteratively select candidate sensor locations, from the plurality of candidate sensor locations, based on the ranking, wherein the iteratively selecting comprises, in each iteration:
selecting for gas emission detection sensor placement a candidate sensor location, from the plurality of candidate sensor locations, having a highest ranking; and
removing at least the selected candidate sensor location from the plurality of candidate sensor locations; and
output the selected sensor locations.

20. The apparatus 19, further comprising:
a network interface or user interface configured to obtain the wind rose distribution data; and
a display configured to output the selected candidate sensor locations.
